(19) United States
(12) Patent Application Publication
(10) Pub. No.: US 2017/0327959 A1
(43) Pub. Date: Nov. 16, 2017

(54) METHOD AND APPARATUS FOR CONVERTING CARBON DIOXIDE

(71) Applicant: Gensoric GmbH, Rostock (DE)

(72) Inventors: Lars KRÜGER, Rostock (DE); Christoph HERZ, Rostock (DE); Martin SCHÖNHOFF, Rostock (DE)

(21) Appl. No.: 15/527,465

(22) PCT Filed: Nov. 17, 2015
(86) PCT No.: PCT/DE2015/100492

§ 371 Date: May 17, 2017

(30) Foreign Application Priority Data
Nov. 17, 2014 (DE) 10 2014 016 894.8

(51) Int. Cl. C25B 3/04; C12P 7/04; C12P 7/24

(52) U.S. Cl. CPC C25B 3/04; C12P 7/24; C12P 7/04; F24D 2200/02

(57) ABSTRACT

The invention relates to a method for preparing a hydrocarbon by reducing CO₂, wherein CO₂ is reduced to a hydrocarbon with the aid of a directly heated electrode. A device for carrying out a corresponding method, a corresponding power plant and a system comprising said power plant and a vehicle with a combustion engine are also objects of the invention. The method and device may, e.g., be used as a micro-energy system for decentralized energy supply.

The invention relates to a method for preparing a hydrocarbon by reducing CO₂, wherein CO₂ is reduced to a hydrocarbon with the aid of a directly heated electrode. A device for carrying out a corresponding method, a corresponding power plant and a system comprising said power plant and a vehicle with a combustion engine are also objects of the invention. The method and device may, e.g., be used as a micro-energy system for decentralized energy supply.

### Our Vision—Residential Small Scale Fuel Generation—Independence, Decentralization, and Commercial Viability

Through our solution, in the next five to seven years, every household can contribute to a CO₂ neutral economy and attain energy independence from external gas or energy supply for heating homes or buildings. This will be achieved by the use of alternative fuels and an efficient energy storage system based on CO₂-to-chemical utilization.

For this aim, a structural change of existing infrastructure is however not needed. In fact, our system will preferably be compatible with existing heating systems and make use of educts which are extensively available at low cost: ambient air, water, and excess energy from renewable sources.

### Aspired Project Result—a Micro Energy System for Decentralized, Domestic Use

This aim is in particular reached by the subject matter of the claims.

One object of the invention therefore in particular is a method for producing a hydrocarbon by reducing CO₂, comprising steps wherein CO₂ is reduced to a hydrocarbon with the aid of a directly heated electrode.

The reduction may occur enzymatically.

The reduction may be carried out in several steps, wherein the reduction in at least one, preferably, in all steps, is catalyzed by an enzyme associated with a directly heated electrode.

On the one hand, immobilization of enzymes favors a better positioning of the enzyme on the electrode surface for better uptake of electrons, and thus allows for improved activity and rate of generation of the product. On the other hand, the enzymes are also stabilized by the immobilization. The macromolecules are always subject to conformation changes, which, to a certain part, contribute to their catalytic activity. Influences from the outside (e.g., temperature or radiation) can however also lead to irreversible conformation states, aggregation of the enzymes can occur, wherein the function of the biocatalyst is abrogated. Immobilization helps to bind the enzymes locally, so that no inactive conformation states are generated any more, and prevents aggregation.

For example, an enzyme can be immobilized in alginate on a carbon fabric and used for reducing CO₂ to formic acid at a working electrode (Wagner A. Enzyme Immobilization on Electrodes for CO₂ Reduction. 2013. Institute of Physical Chemistry). An immobilization in alginate stabilizes the enzymes, however, their positioning in relation to the electrode is not directionally.

Alternatives to immobilization in alginate for a better positioning of the enzymes on electrodes are, e.g., immobilization on carbon nanotubes via functional groups of the enzymes, on gold surfaces via covalent sulfide bonds or on Ni materials via histidine residues on the enzymes. For this, suitable amino acids which can directly bind to support material of the heated electrode may be added to the enzyme during production.

A plurality of steps, preferably, all steps, may be catalyzed by enzymes which are each associated with an electrode directly heated to a temperature optimal for the respective reaction. Preferably, the temperature of the respective electrode is optimized with regard to the matter conversion rate of the respective enzyme, it is however also possible to choose a lower temperature if otherwise a sufficient stability of one or more enzymes is not warranted. If, for a good total conversion not all reactions require a raised temperature compared to the ambient temperature or the reactor temperature, it is also possible that one or more of the enzymes are not associated with a directly heated electrode. For example, the temperature of an electrode associated with a formate dehydrogenase from Candida spp. may be adjusted to 35-40° C., in particular, about 37-38° C.

Also, a plurality of steps, preferably, all steps, may be catalyzed by enzymes which are associated with the same directly heated electrode. Preferably, the temperature of this electrode is optimized with regard to the total conversion rate of matter, it is however also possible to choose a lower temperature if otherwise a sufficient stability of one or more enzymes is not warranted.

The reduction may be carried out in several steps, wherein the reduction in at least one, preferably, in all steps, is catalyzed by an enzyme which at the same time oxidizes a cofactor which is regenerated at a directly heated electrode, wherein the cofactor is selected from the group comprising NADH, NADPH, and FADH.

The reduction may be catalyzed by formate dehydrogenase, aldehyde dehydrogenase and/or alcohol dehydrogenase.

Suitable enzymes are commercially available, however, they can also be further optimized (Felber S., Optimierung der NAD-abhangigen Formiatdehydrogenase aus Candida boidinii für den Einsatz in der Biokatalyse. 2001). As formate dehydrogenase, e.g., an enzyme from Candida spp., in particular from Candida biodinii (e.g., from Sigma-Adrich) can be selected, which has a temperature optimum of 35-40° C. and works with NADH as cofactor. An enzymatic regeneration of the cofactor NADH at a directly heated electrode is optionally possible.

With regard to a continuing bioelectrocatalytic methanol synthesis from formic acid e.g., an aldehyde dehydrogenase from Pseudomonas sp. and an alcohol dehydrogenase from Saccharomyces sp. may be used, which can advantageously be combined with the formate dehydrogenase from Candida spp., in particular, because these enzymes are also dependent on NADH. For the regeneration of the cofactor, e.g., a diaphorase from Pyrococcus sp. may be used.

In said process, CO₂ may be transformed to bicarbonate by a carboanhydrase, wherein, optionally, said carboanhydrase is associated with a directly heated electrode.

Alternatively, the reduction may occur non-enzymatically at a heated electrode, wherein said electrode preferably comprises a material selected from the group comprising platinum, copper, titan, ruthenium and combinations thereof.

Different directly heatable electrodes, wherein the heating element consists of the electrode only, i.e., the temperature increase is only derived from the electrode and is not transferred from the electrolyte solution to the electrode, are known in the art.

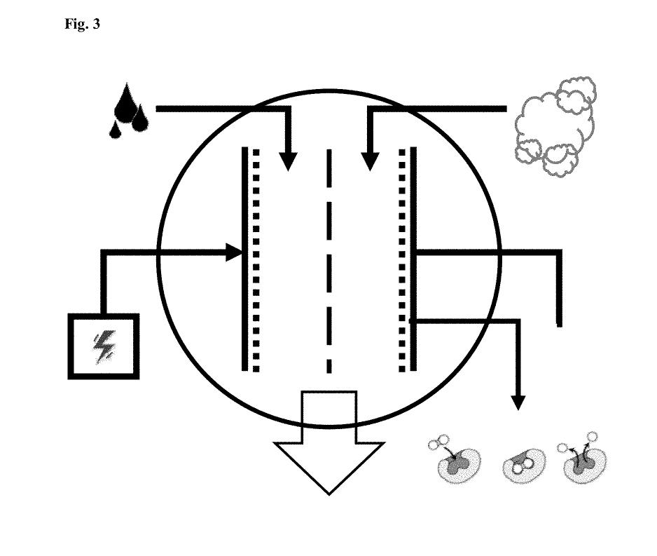

A direct electrical heating of the working electrode can, according to the state of the art, be rendered possible by a so called symmetric order or by special filter circuits. One variant of a directly heated working electrode comprises a third contact for the connection with the electrochemical measuring instrument exactly in the middle between the two contacts for the supply of heating current. By this order, interfering influences of the heating current on the measured signals are prevented. One disadvantage in this is the complex structure with three contacts per working electrode, the thermal interference by the third contact which diverts warmth, and the complicated miniaturization. In a variant preferred according to the invention, a symmetric contact via a bridge circuit is employed, which allows for a direct heating (Wachholz et al., 2007, Electroanalysis 19, 535-540, in particular FIG. 3; dissertation Wachholz 2009). In this, the working electrode can be formed such that the temperature distribution on the surface of the electrode is homogenous (DE 10 2004 017 750). DE 10 2006 006 347, describes advantageous directly heatable electrodes.

In the process according to the invention, the directly heated electrode may have the form of a spiral or a helix or net or plane, in particular as disclosed in DE 10 2014 114 047.

Suitable directly heated electrodes are e.g., available from Gensoric GmbH (Rostock, DE).

The directly heated electrode consists of an electrode material selected from the group comprising carbon, in particular, vitreous carbon or graphite, a precious metal, in particular, gold or platinum, an optically transparent conductive material, in particular stannic oxide doped with indium, copper, stainless steel and nickel.

One object of the invention also is a device in which a method according to any of the preceding claims is carried out or which is suitable for carrying out said method, said device comprising two electrodes and a membrane for separating the anodic and cathodic reaction, or consisting thereof.

A plurality of reaction vessels may be run in parallel, which can together produce the reaction product.

The device may be constructed as a single use reactor or a reactor suitable for recycling and can be used accordingly.

One object of the invention also is a device for preparing a hydrocarbon by fixing CO₂, comprising
a) a directly heated electrode, with which, preferably, at least one enzyme capable of catalyzing a step in the reduction of CO₂ to a hydrocarbon is associated, or, preferably, at least one cofactor capable of interacting with an enzyme capable of catalyzing a step in the reduction of CO₂ to a hydrocarbon is associated, and
b) a device for introducing gaseous CO₂, suitable for introducing the CO₂ into a reaction compartment in which it can contact the directly heated electrode.

Said device typically comprises a further electrode and a membrane for separating the anodic and cathodic reaction.

A device according to the invention may comprise 1-10 000 reaction vessels with directly heated electrodes, preferably, 100-5 000 or 500-2 000 or 800-1 200 reaction vessels. A plurality of reaction vessels can be run in parallel and can in total generate the reaction product.

The device may be constructed as a single use reactor or a reactor suitable for recycling and can be used accordingly.

The gaseous CO₂ can be used from the ambient air or it can be purified or used in concentrated form, e.g., from a gas cylinder.

One object of the invention also is a power plant for providing energy in the form of electric energy and/or a hydrocarbon, comprising
i) an energy source, preferably a regenerative energy source, e.g., based on photovoltaics, hydrodynamic energy or wind-energy, preferably, photovoltaics,
ii) the device according to the invention, wherein the energy required for the preparation of a hydrocarbon is derived from the energy source i),
iii) a hydrocarbon storage device, and
iv) optionally, a hydrocarbon fuel cell for producing electric energy, or
v) optionally, a device for burning hydrocarbon for preparation of warm water or thermal energy for heating of buildings or apartments.

One object of the invention also is a system comprising a power plant according to the invention and a vehicle selected from the group comprising car, bus and motorcycle, wherein the vehicle is equipped with an engine suitable for, preferably, optimized for, combustion of a hydrocarbon, preferably, methanol.

In the context of the invention, the hydrocarbon is selected from the group comprising methanol and methane and formic acid and formaldehyde, preferably, methanol.

Not only for residential homeowners who wish to be more independent form centralized energy supply and who wish to make their existing renewable energy system (RES) more profitable in an environment with unknown prices and incentives, the result of the project is a micro energy system (MES) which helps in storing the surplus energy of the RES and in securing the energy supply, mainly for heating, on a small scale. In contrast to other CO₂ utilization and Power-to-X storage approaches, the application is particularly suited for residential application, as it works under mild ambient conditions (no high pressure, no high temperatures) and is designed for seamless integration into existing infrastructure (heating systems). Owing to the selectivity of the underlying novel electro-enzymatic approach, there is no need for electrolytic generation of hydrogen. In fact, all substrates can be used directly from the environment (ambient air, tap water).

Within this project, the electro-biocatalytic conversion of CO₂ and electricity to methanol through a cascade of enzymatic reactions is the key process. The resulting methanol will be used as fuel for the heating system. No external storage or infrastructure is needed.

### Commercial Viability

### For the End User,

our system will be an attractive alternative to centralized gas or energy supply. The aspired annual cost structure for the system and the consumables shall be comparable to the cost for annual gas consumption (Reference: 10 Ct/kWh Natural Gas in Germany in ˜5-10 years). In addition, our solution enables the efficient storage of energy generated by renewable (e.g. solar) sources.

### For the Commercialization Partner, i.e., Strategic Partners,

the need for revolving purchases of consumables (enzyme reactor) in addition to the single sale of the MES, offers an attractive potential for sale, exceeding 1 Bln £ p.a. in Germany by 2030.

In the context of the invention, “a” means “one or more”, unless specified otherwise.

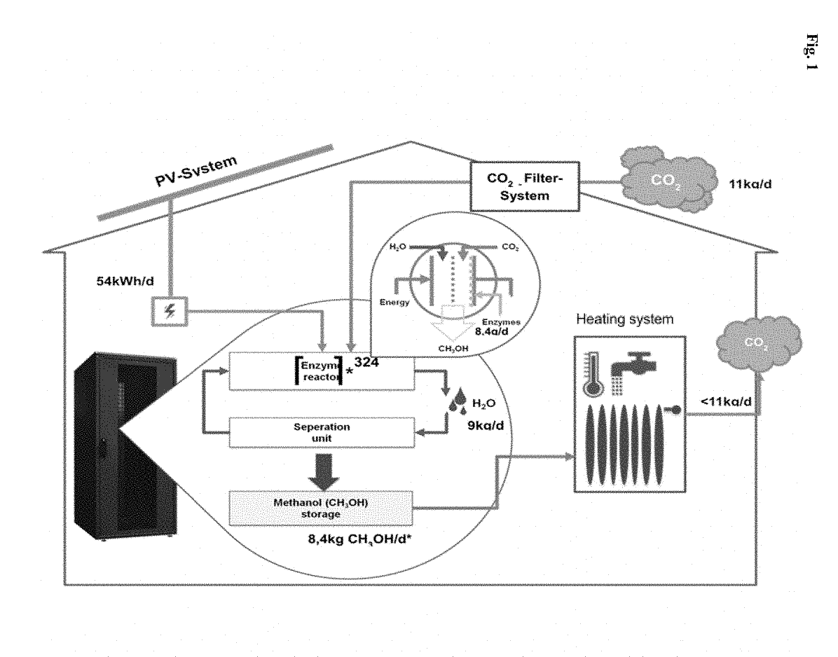

FIG. 1 Scope of the project.

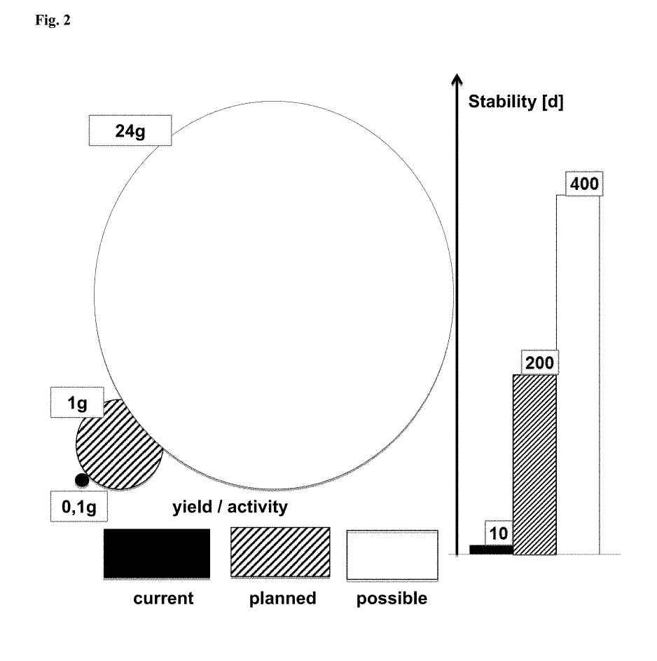

FIG. 2 Planned result of the project activity: Improvement of productivity and stability of the enzyme

FIG. 3 Core technology—Electro-enzymatic reactor, in which CO₂, H₂ from H₂O and electricity are transformed into high-value fuel such as CH₃OH (methanol).

### EXAMPLES

### Project Description

1. Core Technology Development Activities I—Electro-Enzymatic Reactions

Our key reaction will be the electrochemical formation of a C1-organic molecule, like methanol (CH₃OH), in an enzyme-cascade over several steps, carried out at conducting and directly heated electrodes.

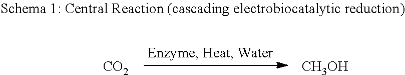

This approach is new, e.g., due to the following features:
mild conditions—no high pressure or high temperatures needed,
high selectivity due to enzymatic conversion;
   No purification/concentration of high volume streams of ambient air
   No electrolysis in H₂O-conversion to produce H₂ directly from water (producing O₂)
Higher reaction velocity, higher turnover without significant or without degradation of enzymes through the use of directly heated electrodes in comparison to non-heated electrodes; and/or
Direct control of turnover and enzyme activity through integrated electrochemical measurements of turnover and temperature is possible

These features distinguish our approach from further CO₂ utilization techniques and make it particularly suitable for applications at a small scale.

Key activities in this project will target the enhancement of the enzymes' yield and stability, i.e, elongation of lifetime. The current status and planned project result are shown in FIG. 2.

The planned activities to attain these aims are:
Increase enzyme stabilities and activity (by factors of 100 and 30, respectively)
Decrease enzyme manufacturing costs (aim: <10 £/g)

Similar measures have proven to be successful in prior projects of 3 years duration.

There are examples of scientifically proven developments, where enzymes were enhanced by using heated electrodes or heated reaction media. These enzymes are typically derived from thermophilic organisms and catalyse the partial reaction in the NADH cycle [McPherson, I. J. und Vincent, K. A.; Electrocatalysis by hydrogenases: lessons for building bio-inspired device. Journal of the Brazilian Chemical Society, 2014].

2. Core Technology Development Activities II—Electro-Enzymatic Reactor

The key reaction cascade is carried out in a specifically designed reactor. Due to our intended business model, the main aim of this project is the development and realisation of a disposable electro enzymatic reactor in a cost-effective manner (assembly, placement of enzymes, wiring). However, a recyclable reactor may also be used, in which, e.g., after a decrease in the efficiency, after a purification, new enzymes are associated with the electrodes.

The reactor will contain directly heated electrodes onto which the enzymes will be immobilized in a way that electrons from the electrical energy source can be transferred to the electro-biocatalytic reaction. This will be achieved by using big area electrodes in beaker glasses or by modifying the inside of the tube-reactors.

3. Core Technology Development Activities III—System Integration

According to the project's overall aim, the core technologies will preferably be integrated into a standalone system which can be integrated into existing domestic heating infrastructure.

The basic functional system: an optimized enzyme-cascade is able to produce x gram product within y hours by using z milligram enzyme-catalyst. It is our goal to produce 5 kg of methanol per day for use in existing infrastructure, e.g., heating systems. Owing to the complexity of scaling up bio-catalytic reactions, we will focus on a discrete scale-up strategy: just to increase the number of (disposable) parallel-running electro-biocatalytic reactors. According to current designs, 1.000 parallel reactors will be capable of yielding the intended amount of energy/fuel per day.

The reaction-medium is separated from the product methanol, e.g., by using a pervaporation unit. The reaction medium will be pumped in a cycle, while the methanol is produced and stored inside the device.

4. Comparative Experiments for Bioelectrocatalysis with a Non-Heated Electrode

Before an optimization bioelectrocatalysis was carried out with the HF Thermalab® (Gensoric, Rostock, DE), preliminary experiments for reducing CO₂ to formic acid were carried out with a nonheatable enzyme alginate electrode. For preparing this electrode, 75 mg processed preparation of a formate dehydrogenase from Candida spp. (Candida boidinii) was used (Sigma Aldrich). The enzyme was immobilized in alginate on a carbon fabris used as a working electrode for reducing CO₂ to formic acid (Wagner A. Enzyme Immobilization on Electrodes for CO₂ Reduction. 2013. Institute of Physical Chemistry). As a reference electrode, a silver silver chloride electrode (Ag/AgCl) with 3 M KCl and as counter electrode a 2 mm graphite rod were used. All reactions were carried out at room temperature in 20 mL of a water based buffered electrolyte solution (0.05 M TRIS, pH 7.7) in a 100 mL reactor. For the bioelectrocatalytic synthesis of formic acid, the reactor was continually supplies with gaseous CO₂. Control experiments were carried out in argon saturated electrolyte solutions in the absence of CO₂. The functionality of the enzyme alginate electrode was first tested by cyclovoltammetry, and the reduction peak of CO₂ was determined to be at about −0.8 V.

Then, the synthesis of formic acid was carried out with chronoamperometry (Table 1).

TABLE 1
Bioelectrocatalytic synthesis of formic acid
from CO₂ by means of chronoamperometry.

| | voltage | Concentration of | Amount of |
|---|---|---|---|
| | (vs. Ag/AgCl, | formic acid | formic acid |
| Experiment | 3M Cl⁻) | (after 9 h) | (after 9 h) |
| No. 1 | −1 V | 0.16 mM | 0.15 mg |
| No. 2 | −0.8 V | 0.15 mM | 0.14 mg |

In a first preliminary experiment, with a voltage of −1 V and at ambient temperature, in total 0.15 mg formic acid could be prepared from CO₂ in a bioelectrocatalytic manner. The quantification was carried out by an enzymatic assay of the sample and via HPLC. In a second preliminary experiment, a voltage of −0.8 V was applied. A similar yield of 0.14 mg formic acid from CO₂ was obtained.

5. Optimization of Bioelectrocatalysis at Heated Electrodes

In the further course of the experimental series, the bioelectrocatalytic reduction of CO₂ at heated electrodes was to be optimized. In this, by targeted heating of the electrodes, the effect of the temperature on the catalytic characteristics of the immobilized enzyme was to be analyzed and thus, optimal parameters for the enzyme catalyzed reaction were to be found. The system HF Thermalab™ from Gensoric was used for the experiments.

1 mg enzyme (formate dehydrogenase from Candida sp.) was immobilized through alginate suspension on a heated microelectrode. With this, a series of chronoamperometric measurements at different temperatures (22° C. 30° C., 35° C., 40° C., 45° C.) was carried out. As reference- and counter electrode, the electrodes form the comparative experiments were used. Before the experiments, 1 mg of regenerated cofactor NADH was added and a test of the enzyme microelectrode for bioelectrocatalytic activity with CO₂ was carried out at ambient temperature. In every case, this test was between −320 μA and −330 μA of a chronoamperogram carried out at −0.8 V for 2-3 minutes. During the experiments, the temperature of the electrolyte in the reactor was measured. As a negative control, an experiment with alginate without enzyme on the microelectrode was carried out.

In the comparative experiments, voltages of −1.0 V and −0.8 V were used. As the difference in yield was less than 10%, and the input of energy into the reactor was to be minimized to avoid overpotentials, a voltage of −0.8 V (vs. Ag/AgCl, 3 M Cl⁻) was used.

In all chronoamperometric measurements, an increased electrical current flow could be observed at the start of the measurements, which decreased after about 3 hours, respectively, to a constant level. The electrical current flow of the reaction at an electrode temperature of 40° C. was highest in comparison to other experiments, both for the start and during the course of the further reaction, which allows the conclusion that there are comparatively high reduction rates. In contrast, the chronoamperogram at 22° C. had the lowest electrical current flow of the enzyme electrodes. The course without enzyme for control hardly showed electrical current flow with about −30 μA, further decreasing to about 0 μA in the course of the reaction. During the chronoamperometric measurements, samples were taken from the reactor after 3 h and after 9 h, respectively, which were analyzed for synthesized formic acid via HPLC (Table 2). In all experiments with heated enzyme microelectrodes, formic acid from the reactor continually supplied with CO₂ was already detected after 3 h, wherein the amount approximately doubled after 9 h. In this, at an electrode temperature of 40° C., the highest amount of formic acid was detected, whereas in experiments with other electrode temperatures (22° C., 30° C., 35° C., 45° C.), less formic acid was produced. With the exception of the experiment at 45° C., the yield of product continually increased proportionally to the temperature of the electrode. During the experiments, the temperature of the electrolyte solution in the reactor was continually monitored. Even at the highest applied heating power, at 45° C. electrode temperature, the electrolyte solution in the reactor was constantly at 22° C. (Table 2).

TABLE 2
Bioelectrocatalytic synthesis of formic acid from
CO₂ at different temperatures on heated electrodes.

| | Formic acid | Formic acid | Electrolyte |
|---|---|---|---|
| Electrode | after 3 h | after 9 h | temperature |
| temperature | of reaction | of reaction | in the reactor |
| 22° C. (with enzyme) | n.d.* | 0.05 mg | 22° C. |
| 30° C. (with enzyme) | 0.09 mg | 0.19 mg | 22° C. |
| 35° C. (with enzyme) | 0.13 mg | 0.29 mg | 22° C. |
| 40° C. (with enzyme) | 0.14 mg | 0.30 mg | 22° C. |
| 45° C. (with enzyme) | 0.12 mg | 0.22 mg | 22° C. |
| 22° C. (control | n.d.* | n.d.* | 22° C. |
| without enzyme) | | | |

*n.d.: not detected

### DISCUSSION

In comparative experiments, the synthesis of 0.15 mg formic acid from CO₂ by chronoamperometry (−1 V; vs. Ag/AgCl, 3 M Cl⁻) in the course of 9 h at room temperature in a 100 mL reactor could be shown. In this, the selective catalytic characteristics of the electro enzyme allowed for application of a low voltage (−0.8 V; vs. Ag/AgCl, 3 M Cl⁻) for nearly the same power of synthesis (0.14 mg formic acid in 9 h). In following experiments, the successful synthesis of 0.05 mg formic acid was possible under similar conditions. One essential difference between comparative experiments and the first following experiment at ambient temperature was in the amount of enzyme used as well as the nature of the electrode material. Whereas in the comparative experiment, in total 75 mg of enzyme were immobilized on the carbon textile as bioelectrocatalyst, in the following experiments, a heatable microelectrode from Gensoric was used, wherein, due to the much smaller electrode surface, only 1 mg of enzyme was immobilized.

Accordingly, the yield with the heatable microelectrode in relation to the use of catalyst was clearly higher with 0.05 mg formic acid per mg enzyme in comparison to the comparative experiment with about 0.002 mg formic acid per mg enzyme.

The advantage of electrodes heated for use is that temperature for optimal reaction conditions can be directly adjusted at the electrode surface, and it is not required to maintain temperature in the complete electrolyte solution of each reactor, which improves the balance of energy of electrocatalytic processes of every kind. The temperature in the reactor was continuously monitored during the bioelectrocatalytic synthesis. In this, the temperature was constantly maintained at 22° C. This could either be due to a continuous mixing because of the continuous gas supply to the electrolyte, favoring a transport of warmth to the surroundings. On the other hand, a part of the warmth from the heated electrodes could be directly channeled to the immobilized enzymes, further stimulating their conformation changes.

In further experiments, the rate of synthesis of formic acid was optimized by direct heating of the enzyme microelectrodes. The highest rates of synthesis, with 0.02-0.03 mg/h (in relation to a constant rate of synthesis according to the chronoamperogram after the first 3 hours of the reaction) occurred at 35° C. and 40° C. In comparison, the rate of synthesis of formic acid decreases both with a decrease of the electrode temperature to 22° C. and an increase to 45° C. Probably, both uptake of substrate and delivery of product at the enzyme microelectrode and changes of conformation status of the immobilized enzyme required for execution of the catalytic reaction mechanism were optimal between 35° C. and 40° C., leading to an increase of conversion by the factor 6 compared to ambient temperature (22° C.). It is interesting to note that in the literature, reaction optima of the same enzyme in solution are described to be about 60° C. (Tishkov V et al., Catalytic mechanism and application of formate dehydrogenase. Biochemistry (Moscow), 2004, 69(11):1252-1267).

In total, at the beginning of each experiment, respectively, the strongest flow of current was measured, which indicates that the majority of the reactions at the electrodes occurs in the first hours. This could be confirmed by measuring the concentration of formic acid. After 3 h, about half of the formic acid present in the further experiment after further 6 h had accordingly already been synthesized. The decrease of the reaction rates can be explained with the increasing product concentration in the bioelectrocatalysts. According to this, diffusion effects are responsible for the reaction taking place more quickly in the beginning. Probably, furthermore, the regeneration of NADH is a limiting factor for the bioelectric catalysis. As, in the beginning, there was enough cofactor for reduction of CO₂, the reactions all took place the fastest. In the further course, the cofactor was reduced to isomers which could not anymore be used for the enzymatic reaction, which led to a decreased speed of the reaction. An effective regeneration of the cofactor, e.g., also enzymatic regeneration, can thus increase efficiency of the reaction.

As the experiments with different temperatures were always carried out with the same enzyme microelectrode, it is possible to start from the assumption that the bioelectrocatalytic synthesis of formic acid further constantly rises even beyond the 9 h, as, in the tests before each experiment, degeneration of the immobilized material over the duration of one week was not observed.

1. A method for producing a hydrocarbon by reducing CO₂, comprising reducing CO₂ to a hydrocarbon with the aid of a directly heated electrode.

2. The method according to claim 1, wherein the reduction occurs enzymatically.

3. The method according to claim 1, wherein the reduction is carried out in several steps, wherein the reduction in at least one, preferably, in all steps, is catalyzed by an enzyme associated with a directly heated electrode.

4. The method according to claim 3, wherein a plurality of steps, preferably, all steps, are catalyzed by enzymes which are each associated with an electrode directly heated to a temperature optimal for the respective reaction.

5. The method according to claim 3, wherein a plurality of steps, preferably, all steps, are catalyzed by enzymes which are associated with the same directly heated electrode.

6. The method according to claim 1, wherein the reduction is carried out in several steps, wherein the reduction in at least one, preferably, in all steps, is catalyzed by an enzyme which at the same time oxidizes a cofactor which is regenerated at a directly heated electrode, wherein the cofactor is selected from the group comprising NADH, NADPH, and FADH.

7. The method of claim 1, wherein the reduction is catalyzed by formate dehydrogenase, aldehyde dehydrogenase and/or alcohol dehydrogenase.

8. The method of claim 1, wherein the CO₂ is transformed to bicarbonate by a carboanhydrase, wherein, optionally, the carboanhydrase is associated with a directly heated electrode.

9. The method according to claim 1, wherein the reduction occurs non-enzymatically at a heated electrode, wherein said electrode preferably comprises a material selected from the group comprising platinum, copper, titan, ruthenium and combinations thereof.

10. The method of claim 1, wherein the directly heated electrode has the form of a spiral or a helix or net or plane.

11. The method of claim 1, wherein the directly heated electrode consists of an electrode material selected from the group comprising carbon, in particular, vitreous carbon or graphite, a precious metal, in particular, gold or platinum, an optically transparent conductive material, in particular stannic oxide doped with indium, copper, stainless steel and nickel.

12. A device in which a method of claim 1 is carried out or which is suitable for carrying out said method, said device comprising two electrodes and a membrane for separating the anodic and cathodic reaction.

13. The device according to claim 12, wherein a plurality of reaction vessels are run in parallel, which can together produce the reaction product.

14. The device according to claim 12, which is constructed as a single use reactor or a reactor suitable for recycling.

15. A device for preparing a hydrocarbon by fixing CO₂, comprising
a) a directly heated electrode, with which, preferably, at least one enzyme capable of catalyzing a step in the reduction of CO₂ to a hydrocarbon is associated, or, preferably, at least one cofactor capable of interacting with an enzyme capable of catalyzing a step in the reduction of CO₂ to a hydrocarbon is associated, and
b) a device for introducing gaseous CO₂, suitable for introducing the CO₂ into a reaction compartment in which it can contact the directly heated electrode,
wherein the device optionally is a device according to claim 12.

16. A power plant for providing energy in the form of electric energy and/or a hydrocarbon, comprising
i) an energy source, preferably a regenerative energy source, e.g., based on photovoltaics,
ii) the device according to claim 12, wherein the energy required for the preparation of a hydrocarbon is derived from the energy source i),
iii) a hydrocarbon storage device, and
iv) optionally, a hydrocarbon fuel cell for producing electric energy, or
v) optionally, a device for burning hydrocarbon for preparation of warm water or thermal energy for heating of buildings or apartments.

17. A system comprising a power plant according to claim 16 and a vehicle selected from the group comprising car, bus and motorcycle, wherein the vehicle is equipped with an engine suitable for, preferably, optimized for, combustion of a hydrocarbon, preferably, methanol.

18. The method of claim 1, device, power plant or system according to any of the preceding claims wherein the hydrocarbon is selected from the group comprising methanol and methane and formic acid and formaldehyde.

19. The device of claim 1, wherein the hydrocarbon is selected from the group comprising methanol and methane and formic acid and formaldehyde.

20. The power plant of claim 1, wherein the hydrocarbon is selected from the group comprising methanol and methane and formic acid and formaldehyde.

21. The system of claim 1, wherein the hydrocarbon is selected from the group comprising methanol and methane and formic acid and formaldehyde.
